(19) United States
(12) Patent Application Publication
(10) Pub. No.: US 2003/0077264 A1
(43) Pub. Date: Apr. 24, 2003

(54) Antimicrobial blood treatment using allicin and related compounds

(76) Inventor: Laura L. Goodrich, Lakewood, CO (US)

Correspondence Address: GREENLEE WINNER AND SULLIVAN P C, 5370 MANHATTAN CIRCLE, SUITE 201, BOULDER, CO, 80303, US

(21) Appl. No.: 10/247,262

(22) Filed: Sep. 18, 2002

Related U.S. Application Data
(60) Provisional application No. 60/323,669, filed on Sep. 20, 2001.

(51) Int. Cl. A61K 45/00; A61K 35/78

(52) U.S. Cl. 424/93.72; 424/93.73; 424/754

(57) ABSTRACT

Methods and compositions prolonging the storage life and/or increasing the safety of a blood product, such as whole blood, red blood cells, white blood cells, platelets, serum and aqueous additive solutions for storage of such blood products are provided. Storage solutions of this invention comprise a composition selected from the group consisting of garlic extract, allicin, other microorganism-growth-inhibiting compounds derived from garlic, and analogs and derivatives of allicin and said other compounds, in an amount effective to inhibit growth of at least one selected microorganism which is a bacterium, virus, fungus or parasite. The storage additive solutions of this invention can increase platelet storage life by at least about 20%, preferably at least about 40%.

### CROSS-REFERENCE TO RELATED APPLICATIONS

This application claims priority to U.S. provisional application No. 60/323,669 filed Sep. 20, 2001, which is incorporated herein by reference in its entirety to the extent not inconsistent herewith.

### BACKGROUND

Garlic (Allium sativum) is a popular cooking spice and has been known as a folk remedy for centuries. Its use was described by Virgil in the Second Idyll as a treatment for snake bite and by Hippocrates for treating pneumonia and suppurating wounds. It has also been used for treating gastric catarrh, dysentery, typhoid and cholera. (E. D. Wills (1956) “Enzyme Inhibition by Allicin, the Active Principle of Garlic,” Biochem J. 63:514-519.) It has alterative, stimulant, diaphoretic, expectorant, antiseptic, antibiotic, antispasmodic, cholagogue, vulnerary, vermifuge, antibacterial, and antifungal properties. (“Allium sativum—Garlic (Liliaceae),” The Herbalist, newsletter of the Botanic Medicine Society, 1988) downloaded Dec. 5, 2000 from www.ibiblio.org.) Aqueous extracts (25% w/v) of garlic are bacteriostatic and bactericidal against a number of bacteria on blood agar and at further dilutions (down to 1/32, and in one case 1/64, of the original extract). (I. Elamin et al. (1983), “The Antimicrobial Activity of Garlic and Onion Extracts,” Pharmazic 38:747-748.)

Other health concerns for which garlic is recommended are atherosclerosis, candidiasis, hypertension and hypoglycemia. It is known as having fibrinolytic properties and inhibits blood platelet aggregation as well as reducing plaque in arteriosclerosis therapy. (“Garlic,” Whole Health Discount Center website, downloaded Dec. 5, 2000 from www.health-pages.com/ga/. Garlic extract has been shown to protect against cardiovascular disease as a result of inhibiting platelet aggregation. (K. Rahman and D. Billington (2000), “Dietary Supplementation with Aged Garlic Extract Inhibits ADP-Induced Platelet Aggregation in Humans,” J. Nutr. 130:2662-2665.) In the People's Republic of China, it is administered orally and intravenously to treat cryptococcal meningitis, and has been shown to possess antiviral activity against influenza B and herpes simplex viruses but not against Coxsackie B1 virus. (Y. Tsai et al. (1985), “Antiviral Properties of Garlic: In vitro Effects on Influenza B Herpes Simplex and Coxsackie Viruses,” Planta Medica 51:460. It has also been used in combination with other herbs for treatment of psoriasis, rheumatism and asthmatic dyspnea. (U.S. Pat. No. 5,165,932 issued Nov. 24, 1992 to Horvath for “Therapeutical Compositions Against Psoriasis.”)

Fermented with rice bran and Aspergillus, then extracted with ethanol, garlic is said to be useful as a coating agent for treating diseases of trichophytosis. (PCT Publication 88/04933 dated Jul. 14, 1988 for “Specially Processed Garlic Product” (Abstract)). Enzymatically-deactivated and fermented with Aspergillus and/or Monascus, it is known as a prophylactic or therapeutic agent for diabetes, hepatic disease, cancer, immunopathy, and hyperemia. (U.S. Pat. No. 6,146,638 issued Nov. 14, 2000 to Kakimoto et al. for “Fermented Garlic Composition.”) Extracts have been recommended for inhibiting apoptosis. (U.S. Pat. No. 5,635,187 issued Jun. 3, 1997 to Bathurst et al. for “Compositions which Inhibit Apoptosis, Methods of Purifying the Compositions and Uses Thereof.”) A composition made by combining extract of garlic with S-allylcysteine is said to be useful in controlling hepatopathy and oncogenesis. (U.S. Pat. No. 5,093,122 issued Mar. 3, 1992 to Kodera for “Method for Preparing an S-Allylcysteine-containing Composition.” Garlic and extracts have been orally administered for treating and preventing cardiovascular diseases such as myocardial infarction, stroke and multiple arteriosclerosis by reduction of high levels of plasma homocysteine. (U.S. Pat. No. 6,129,918 issued Oct. 10, 2000 to Amagase for “Method and Pharmaceutical Composition for Reducing Serum Homocysteine Concentration.”) U.S. Pat. No. 5,705,152 issued Jan. 6, 1998 to Plummer for “Antimicrobial Composition” discloses the use of dried garlic powder in combination with non-pathogenic microorganisms useful for combating pathogenic microorganisms in animal gastrointestinal tracts.

The active principles present in garlic have been found to be mainly sulfur-containing compounds. The principal component of a colorless oil obtained from steam distillates of garlic extracts was shown to be a sulfur compound, C₆H₁₀S₂O, named allicin (thio-2-propene-1-sulfinic acid S-allyl ester, or alternatively, 2-propene-1-sulfinothioic acid S-2-propenyl ester, or diallyl thiosunfinate). (C. J. Cavallito and J. H. Bailey (1944), “Allicin, the antibacterial principle of Allium sativum. I. Isolation, physical properties and antibacterial action. J. Am. Chem. Soc. 66:1944-1952; and C. J. Cavallito et al. (1944) “Allicin, the antibacterial principle of Allium sativum. II. Determination of the chemical structure. J. Am. Chem. Soc. 66:1952-1954.) The structure of allicin is:

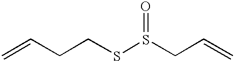

Allicin is a colorless, volatile liquid with a pungent odor, a water solubility of about 2%, moderate solubility in hexane and high solubility in organic solvents more polar than hexane. (“Garlic: The Science and Therapeutic Application of Allium sativum L. and Related Species”(Second Edition, 1996) (H. P. Koch and L. D. Lawson, eds.) pp. 56-57). It is responsible for most of the smell of garlic. A garlic bulb exhibits little or no odor until it is cut or crushed. The intact garlic clove does not contain allicin but rather its odorless precursor alliin ((+)(S)-allyl-L-cysteine sulfoxide) that is converted to allicin, pyruvate and ammonia by a C-S lyase present in the garlic plant termed allicin lyase or alliinase. Alliin and alliinase are found in different compartments of the garlic clove. The cutting or crushing of the clove enables the enzyme to come into contact with the precursor of allicin. (PCT Publication WO 97/39115 dated 23 Oct., 1997, D. Mirelman et al. “Immobilized Alliinase and Continuous Production of Allicin.”) Allicin is an unstable compound, having a half-life of sixteen hours at 23° C., and in water solution at 0.1-2 mg/ml at this temperature of 30-40 days. (“Garlic”, Koch and Lawson eds., supra, p. 58.) Its major biological effects have been attributed to antioxidant activities and to rapid reactions with thiol-containing proteins. (A. Rabinokov et al. (1998), “The mode of action of allicin: trapping of radicals and interaction with thiol containing proteins,” Biochimica et Biophysica Acta 1379:233-244.)

The synthesis of allicin was described in U.S. Pat. No. 2,508,745 issued May 23,1950 to C. J. Cavallito et al. for “Hydrocarbon Esters of Hydrocarbonylthiosulfinic Acids and Their Process of Preparation.”

Other components of garlic and their degradation products are described in L. D. Lawson et al. (1991), “Identification and HPLC Quantitation of the Sulfides and Dialk(en)yl Thiosulfinates in Commercial Garlic Products,” Planta Med. 57:363-370.

C. J. Cavallito and J. H. Bailey (1944) (I., supra) showed in cylinder-plate tests using meat extract broth at pH 6.8 or 0.5% dextrose-veal infusion broth that allicin at dilutions as great as 1:25 prevented growth of a number of bacteria. Allicin has been shown to exhibit a wide spectrum of antibacterial activity against gram negative and gram positive bacteria, including Escherichia coli, antifungal activity against Candida albicans, antiparasitic activity including against Entamoeba hisiolytica and Giardia lamblia, and antiviral activity. (S. Ankri and D. Mirelman (1999), “Antimicrobial properties of allicin from garlic,” Microbes and infection 2:125-129; S. Ankri et al. (1997), “Allicin from Garlic Strongly Inhibits Cysteine Proteinases and Cytopathic Effects of Entamoeba histolytica” Antimicrobial Agents and Chemotherapy, October:2286-2288. See also D. Mirelman et al., “Pathogenesis of the Parasite Entamoeba histolytica,” downloaded from http://bioinfo.weizmann.ac.il on Dec. 5, 2000.) However, mammalian cells are protected by the peptide glutathione which acts to restore activity of affected enzymes. (“A Garlic Charm Against Stomach Bugs,” Academic Press Daily Insight, downloaded Dec. 5, 2000 from www.apnet.com/inscight/10141997/graphb.htm.)

Enzymes of the succinic oxidase system are inhibited by allicin, however, cysteine, glutathione, and 2:3-dimercaptopropanol) are protective agents, and serum has a weak protective action. Allicin is known to inhibit alkaline phosphatase and invertase, urease, succinic dehydrogenase, lactate dehydrogenase, tyrosinase, peroxidase, papain, amylase, xanthine oxidase, choline oxidase, levokinase, cholinesterase, glyoxylase, triose phosphate dehydrogenase. Some of this inhibition occurs in a pH-dependent manner. (E. D. Wills (1956), “Enzyme Inhibition by Allicin, the Active Principle of Garlic,” Biochem J. 63:514-520; Chem. Abstr. 50 (1956) 15612.)

Allicin was shown to have a minimum inhibitory concentration against Helicobacter pyloriof 4.0 μg/ml in medium. (E. A. O'Gara et al. (2000), “Activities of Garlic Oil, Garlic Powder, and Their Diallyl Constituents against Helicobacter pylori,” Applied and Environmental Microbiology, May:2269-2273). U.S. Pat. No. 5,321,045 issued Jun. 14, 1994 to Dorsch et al. for “Method and Composition for the Treatment of Inflammatory Conditions using Thiosulphinic Acid Derivatives” reports that intravenous administration of compounds including allicin at 0.5-500 mg/kg provides anti-inflammatory activity. R. S. Feldberg et al. (1988), “In Vitro Mechanism of Inhibition of Bacterial Cell Growth by Allicin,” Antimicrobial Agents and Chemotherapy, 32:1763-1768) discloses bacteriostatic concentrations of allicin (0.2 to 0.5 mM) in Luria broth for Salmonella typhimurium. A. D. Kaye et al. (July, 2000), “Analysis of responses of garlic derivatives in the pulmonary vascular bed of the rat,” J. Appl. Physiol. 89:353-358) report that allicin and related compounds are vasodilators.

U.S. Pat. No. 4,917,921 issued Apr. 17, 1990 to Hermes for “Antithrombogenic and Antibiotic Composition and Methods of Preparation Thereof” discloses antithrombogenic and antibiotic polymeric coatings made from copolymerization of 2-vinyl-4H-1,3-dithiin (a component of garlic) and N-vinyl pyrrolidone. U.S. Pat. No. 4,665,088 issued May 12,1987 to Apitz-Castro et al. for “(E-Z)-4,5,9-Trithiadodeca-1,6,11-triene-9-oxides” discloses the use of compounds such as (E,Z)-ajoene as antithrombotic agents. H. Yoshida et al. (1999), “An Organosulfur Compound Isolated from Oil-Macerated Garlic Extract,” Biosci. Biotechnol. Biochem. 63:588-590 discloses anitmicrobial effect of E-4,5,9-trithiadeca-1,7-diene-9-oxide isolated from garlic. H. Yoshida et al. (999), “Antimicrobial Activity of the Thiosulfinates Isolated form Oil-Macerated Garlic Extract,” Biosci. Biotechnol. Biochem. 63:591-594, disclose antimicrobial activities of 2-propene-1-sulfinothioic acid S-(Z,E)-1-propenyl ester, 2-propenesulfinothioic acid S-methyl ester, and methanesulfinothioic acid S-(Z,E)-1-propenyl ester.

J. C. Harris et al. (December, 2000), “The microaerophilic flagellate Giardia intestinalis: Allium sativum (garlic) is an effective antigiardial,” Microbiology 146:3119-3127, suggest that the antimicrobial properties of garlic are due to metabolic breakdown products of allicin (diallyl disulphide and diallyl sulphide) rather than allicin itself.

Ajoene is another compound formed as garlic is crushed. Alliin in the garlic comes into contact with allinase in the cell wall to form allicin. Then in the presence of a polar molecule such as a lower alcohol or even water, allicin forms ajoene, which can be administered, e.g. interarterially, for treatment of inflammation. (See U.S. Pat. No. 6,177,475 issued Jan. 23, 2001 to Tatarintsev et al. for “Methods of Using Integrin Modulators for Treatment of Inflammation.” Tatarintsev et al. have also disclosed that ajoene can be administered to a patient for inhibiting integrin-mediate cell-cell fusion (U.S. Pat. No. 5,981,602 issued Nov. 9, 1999), for treatment of shock (U.S. Pat. No. 5,968,988 issued Oct. 19, 1999), for treatment of tumors (U.S. Pat. No. 5,932,621 issued Aug. 3, 1999), for inhibiting immune response (U.S. Pat. No. 5,863,955 issued Jan. 26, 1999), and for inhibiting the progression of infection and other pathologies produced by a viral infection (U.S. Pat. No. 5,948,821).

Isoalloxazine and related compounds have been used as antimicrobials as described in: U.S. Pat. No. 6,258,577 issued Jul. 10, 2001; U.S. Pat. No. 6,277,377 issued Aug. 21, 2001; PCT Publications WO 0194349A1 published Dec. 13, 2001 and WO 0196340A1 published Dec. 20, 2001; U.S. Pat. No. 6,268,120 issued Jul. 31, 2001; PCT Publication WO 0243485A1 published Jun. 6, 2002; and/or U.S. Patent Publication No. 2001/0024781A1 published Sep. 27, 2001, which concern methods and apparatuses for blood and blood product decontamination using isalloxazine and related compounds.

While the antimicrobial effects of allicin and the foregoing related compounds have been described in the literature, these compounds do not appear to have been used as additives to blood, blood products or blood storage solutions to delay growth of microorganisms or kill them.

All publications referred to herein are incorporated herein by reference to the extent not inconsistent herewith.

### SUMMARY

Blood products are typically removed from a donor, separated into components (platelets, plasma, and red blood cells), stored, and used for therapeutic purposes by administration back to the donor or to another recipient. Due to the growth of microorganisms in stored platelets over time, they cannot typically be stored under normal storage conditions for periods longer than about five days. Normal storage conditions for platelets include storage at a temperature of about 22° C. with agitation (preferably on a shaker table at about 72 cycles per minute). In the 1970s, government regulation allowed platelet storage for up to seven days, but occurrence of bacterial infection/contamination caused this period to be reduced to no more than five days.

Other blood products have varying safe storage periods. For example, red blood cells can be stored for up to 42 days at 4° C. in additive solutions compliant with government regulations, but bacteria such as Yersinia entercolitica will still grow. Table 1, taken from the American Association of Blood Banks (AABB) Technical Manual, 13^{th} Edition, 2000, provides storage conditions and expiration dates for a number of blood products.

TABLE 1

| Category | Expiration |
|---|---|
| Whole Blood | ACD¹/CPD²/CP2D³ - 21 days |
| Whole Blood Modified | ACD/CPD/CP2D - 21 days |
| | CPDA-1⁴ - 35 days |
| Whole Blood Irradiated | Original outdate (see outdates |
| | above per anticoagulant) or 28 |
| | days from date of irradiation, |
| | whichever is sooner |
| Red Blood Cells (RBCs) | ACD/CPD/CP2D - 21 days |
| | CPDA-1 - 35 days |
| RBCs, Additive Solutions | 42 days |
| RBCs, Washed | Time approved by FDA |
| RBCs, Leukocytes Reduced | ACD/CPD/CP2D - 21 days |
| | CPDA-1 - 35 days |
| | Open system - 24 hours |
| | Additive solutions - 42 days |
| RBCs, Rejuvenated | 24 hours |
| RBCs, Rejuvenated, Washed | 24 hours |
| RBCs, Irradiated | Original outdate above or 28 |
| | days from date of irradiation, |
| | whichever is sooner |
| RBCs, Frozen 40% Glycerol | 10 years |
| RBCs, Frozen 20% Glycerol | 10 years |
| RBCs, Open System | 24 hours |
| RBCs, Open System - Frozen | 10 years, 24 hours after thaw |
| RBCs, Frozen - Liquid Nitrogen | 10 years |
| Platelets | 24 hours to 5 days, depend- |
| | ing on collection system |
| Platelets, Pheresis | 5 days |
| Platelets Pooled or in Open System | 4 hours, unless otherwise |
| | specified |
| Platelets, Leukocytes Reduced | 4 hours open system |
| | 5 days closed system |
| Platelets, Pheresis, Leukocytes Reduced | 5 days |
| Platelets, Irradiated | 4 hours open system |
| | 5 days closed system |
| Granulocytes | 24 hours |
| Fresh Frozen Plasma (FFP) | 12 months (−18° C.) |
| | 7 years (−65° C.) |
| FFP, Thawed | 24 hours |
| FFP, Open System - thawed | 24 hours |
| Pooled Plasma, Solvent/detergent-treated | 12 months |
| Pooled Plasma, Solvent/detergent-treated - | 24 hours |
| Thawed | |
| Plasma (frozen within 24 hours) | 12 months |
| Plasma (frozen within 24 hours) Thawed | 24 hours |
| Plasma Thawed | >24 hours, <5 days |
| Plasma Liquid | 5 days after expiration of |
| | RBCs |
| FFP - Donor Retested Thawed | 24 hours |
| FFP - Donor Retested | 12 months |
| Plasma, Cryoprecipitate-Reduced, Thawed | 24 hours |
| Cryoprecipitated Anti-hemophilic Factor | 12 months |
| (AHF) | |
| Cryoprecipitated AHF, Thawed | ASAP or within 4 hours if |
| | open system or pooled, 6 |
| | hours if single unit or pooled |

¹ACD is acid citrate dextrose.
²CPD is citrate phosphate dextrose
³CP2D is citrate phosphate dextrose dextrose
⁴CPDA-1 is citrate phosphate dextrose adenine.

Growth of microorganisms in stored blood products renders the products less safe than fresh blood products for administration to patients. Further, short storage periods give rise to problems in matching supply to demand and cause inefficiencies and extra expense in providing patients with required treatments. To increase the safety of stored blood products and allow a longer storage life for blood products, this invention teaches adding to stored blood products compositions which kill microorganisms, or slow the growth thereof. In one embodiment, the storage life of platelets is increased to more than five days, up to at least about six days, preferably up to about seven days.

This invention also provides an aqueous additive solution (also referred to herein as a “storage solution”) for storage of blood products selected from the group consisting of whole blood, red blood cells, white blood cells, platelets, serum and plasma, said additive solution comprising a composition selected from the group consisting of garlic extract, allicin, other microorganism-growth-inhibiting compounds derived from garlic, and analogs and derivatives of allicin and said other compounds, in amounts effective to inhibit growth of at least one selected microorganism.

The term “derived from garlic” means removal of naturally-occurring microorganism-growth-inhibiting compounds from garlic by extraction or other means known to the art. Such compounds need not be isolated in pure form (but may be isolated in pure form) to be useful in this invention.

This invention also provides a composition comprising a mixture of anticoagulant and a storage-life increasing amount (an amount effective to inhibit growth of at least one selected microorganism) of garlic extract, allicin, other microorganism-growth-inhibiting compounds from garlic, or analogs and derivatives of allicin and said other compounds. Such compositions also comprising a blood product are provided as well. Anticoagulants are known to the art and include acid citrate dexytrose (ACD), citrate phosphate dextrose (CPD), citrate phosphate dextrose dextrose (CP2D), and citrate phosphate dextrose adenine (CPDA-1). Such solutions generally have a pH of around 6.4.

Garlic extract or allicin and derivative compositions of this invention may be added to platelet additive solutions known to the art. Such known platelet additive solutions include those disclosed in U.S. Pat. Nos. 5,908,742; 5,482,828; 5,569,579; 5,236,716; 5,089,146; and 5,459,030. Platelet additive solutions may contain physiological saline solution, buffer, preferably sodium phosphate, and other components including magnesium chloride and sodium gluconate. The pH of such solutions is preferably between about 7.0 and 7.4. These solutions are designed as carriers for platelet concentrates to allow maintenance of cell quality and metabolism during storage, reduce plasma content and extend storage life. A preferred platelet additive solution comprises monobasic sodium phosphate, riboflavin, dibasic sodium phosphate, sodium chloride, sodium ascorbate, potassium chloride, and magnesium chloride.

Compositions of this invention containing garlic extract or allicin and derivative compositions preferably have a pH between about 6.4 and about 7.4.

The allicin and related compositions of this invention may be present in the storage solutions at concentrations sufficient to inhibit the growth of at least a selected microorganism, e.g., from about 10 μg/ml to the solubility of the compounds in the solution, and preferably up to about 3000 μg/ml, more preferably at least about 30 μg/ml. It is preferred that the concentration be minimized when odorous compounds such as allicin are used.

Concentrations of allicin and related compounds sufficient to inhibit growth of selected microorganisms in selected blood products may easily be determined as known to the art and in accordance with the teachings hereof. Preferably such compounds are present in the blood product at a concentration of between about 5 volume percent and about 35 volume percent, preferably about 5 to about 10 volume percent. Blood product is preferably present at a concentration between about 65 and about 95 volume percent. “Growth inhibition” as used herein is measurable growth inhibition as determined by assays known to the art.

Sulfur-containing compounds such a glutathione in the blood product or solution may compete with reactions of compositions of this invention and affect their growth-inhibitory action on microorganisms. The concentration of allicin and related compounds present in blood product should be adjusted to take account of such competing reactions.

Selected microorganisms whose growth it is desired to inhibit when storing blood products include gram negative and gram positive bacteria such as Staphylococcus epidermidis, Yersinia enterocolitica, Esherichia coli, Pseudomonas aeruginosa, Staphylococcus aureus, Klebsiella pneumoniae, Treponema pallidum, Bacillus cereus, Clostridium perfringes, Enterobacter cloacae, Proteus mirabilis, Salmonella cholerasuis, Serratia liquefactions, Serratia marscesens, Francisella tularensis, Streptococcus pyogenes, and Streptococcus mitis; viruses such as Human Immunodeficiency Virus (HIV), Influenza A and B, Hepatitis viruses such as hepatitis B and C, Epstein-Barr virus (EBV), Herpes simplex, pneumonia virus, cytomegalovirus (CMV), and parvovirus; fungi such as Candida albicans, Trichophyton cerebriforme, Trychophyton granulosum and Microsporum canis; protozoa such as Paramecium caudatum, Lamblia intestinalis, Grandia lamblia, Trypanosoma cruzi and Entamoeba histolytica; parasites such as Ascaris strongyloides, Oxyuris, Ancyclostoma caninum, and Necator americanus, Trypanosome spp., Plasmodium spp., and Wucheria bancrofti. Preferred microorganisms are blood-borne pathogens such as HIV, CMV, Hepatitis viruses, plasmodium, trypanosome, Francisella tularensis, and Wucheria bancrofti.

Without wishing to be bound to any particular mechanism of action of allicin and related compounds, applicants believe that the enzyme-inhibiting effects of these compounds may interfere with the ability of microorganisms to metabolize, reproduce, or respirate effectively, such that these compounds have the effect of at least preventing or slowing down growth of these microorganisms if not completely destroying them.

Preferred compounds of this invention are those present in extract of garlic. Garlic extract may be prepared by means known to the art. One procedure is to grind garlic cloves, e.g., in a blender, and mix with saline, preferably 0.9% saline, using about 25 g cloves per 100 ml saline. This is followed by separation of the liquid extract, e.g., by centrifugation or filtration, preferably through a 0.2μ filter. The extract may be stored frozen until used.

Garlic may also be used in this invention in the form of garlic powder such as commercially available garlic powder.

Solutions may be prepared using commercially available allicin, e.g., from LKT Laboratories, Inc., St. Paul, Minn. Solutions containing 1 mg allicin per 100 μl of a carrier composed of 60:40:0.1 methanol:water:formic acid are useful in this invention. Allicin may also be prepared by oxidation of commercially available diallyl disulfide, e.g., with hydrogen peroxide/ glacial acetic acid or with perbenzoic or peracetic acid.

Allicin and other garlic components known to the art to have a growth-inhibiting effect on microorganisms may be isolated from garlic extract or synthetically prepared by means known to the art and used in the compositions and methods of this invention. Analogs and derivatives of such compounds may also be prepared by means known to the art and used in the compositions and methods of this invention.

Some garlic components which have a growth-inhibiting effect on microorganisms and related analogs include methyl propyl sulfide, allyl methyl sulfide, diallyl sulfide, methyl sulfide, diallyl disulfide, dimethyl disulfide, methyl propyl disulfide, dipropyl disulfide, allyl alcohol, allyl mercaptan, diallyl trisulfide, diallyl tetrasulfide, diallyl pentasulfide, diallyl hexasulfide, methy allyl disulfide, methyl allyl trisulfide, methyl allyl tetrasulfide, methyl allyl pentasulfide, methyl allyl hexasulfide, dimethyl disulfide, dimethyl trisulfide, dimethyl tetrasulfide, dimethyl pentasulfide, dimethyl hexasulfide, diethyl disulfide, propyl allyl disulfide, 2-vinyl-4H-1,3-dithiin, 3-vinyl-4H-1,2-dithiin, (E)-ajoene, (Z)-ajoene, allicin, allyl methyl thiosulfinate, (E)-2,3,7-trithiaocta-4-ene-7-oxide, (E)-4,5,9-trithiadeca-1,6-diene-9-oxide, (Z)-2,3,7-trithiaocta-4-ene-7-oxide, (Z)-4,5,9-trithiadeca-1,6-diene-9-oxide, (E)-4,5,9-trithiadeca-1,7-diene-9-oxide, (Z)-4,5,9-trithiadeca-1,7-diene-9-oxide, 2-propene- 1-sulfinothioic acid S-(Z,E)-1-propenyl ester, 2-propenesulfinothioic acid S-methyl ester, and methanesulfinothioic acid S-(Z,E)-1-propenyl ester.

A generalized formula for allicin and related compounds useful in this invention is:

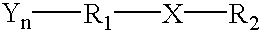

where

R₁ and R₂ are, independently H, C₁-C₂₀ (saturated or unsaturated) alkyl, alkoxy, cycloalkyl or aralkyl;

X is S, SS, SSS, or SOS, and moieties in which one or more of the sulfur atoms are replaced with SO or OSO;

n is 0 or 1; and

Y is R₃ₘXR₄ₚ where

m and p are, independently, 0 or 1; and

R₃ and R₄ are H or C₁-C₆ (saturated or unsaturated) alkyl, alkoxy, cycloalkyl or aralkyl.

C₁-C₆ (saturated or unsaturated) dithiins are also included within the scope of this invention, as are optical isomers and pharmaceutical salts of all the foregoing compounds. Such compounds can be tested for microorganism growth-inhibiting activity by means known to the art and as taught herein.

One class of compounds useful in this invention are those of the above formula in which X is S═O. Another class of useful compounds is those in which m and p are 1, R₃ is methyl, X is S═O and R₄ is C═C—C. The class of compounds wherein R₁ and R₂ comprise double bonds in cis-configuration, spaced apart from sulfur atoms by two carbons, as in allicin, are preferred.

A further class of compounds useful in this invention are those having the formula:

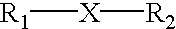

where

R₁ and R₂ are, independently H, or C₁-C₆ (saturated or unsaturated) alkyl;

and optical isomers and pharmaceutical salts thereof.

A further class of compounds useful in this invention are those of the above formula in which R₁ is allyl and R₂ is allyl ethyl, vinyl, or methyl.

The storage solution may also contain isoalloxazine or an isoalloxazine analog, or vitamin E or a vitamin E acetate analog, and/or anticoagulant. Isoalloxazine and its analogs are useful for killing and inhibiting microorganisms. Isoalloxazine analogs include all compounds disclosed in U.S. Pat. No. 6,258,577 issued Jul. 10, 2001; U.S. Pat. No. 6,277,377 issued Aug. 21, 2001; PCT Publications WO 0194349A1 published Dec. 13, 2001 and WO 0196340A1 published Dec. 20, 2001; U.S. Pat. No. 6,268,120 issued Jul. 31, 2001; PCT Publication WO 0243485A1 published Jun. 6, 2002; and/or U.S. Patent Publication No. 2001/0024781A1 published Sep. 27, 2001, all of which are incorporated herein by reference to the extent not inconsistent herewith.

In the methods of this invention, the isoalloxazine and/or related compounds may be added to the solution, before, at the same time, or after addition of the allicin and related compositions of this invention. Preferably such isoalloxazine and/or related compounds are added to blood products at the time of collection or as soon thereafter as practical, i.e., before microorganisms have a chance to proliferate.

This invention also provides methods of prolonging the storage life of a blood product comprising: adding to said blood product a solution comprising at least an amount effective to inhibit growth of a selected microorganism, of a compound selected from the group consisting of allicin and microorganism-growth-inhibiting analogs and derivatives thereof; and storing said blood product. Platelets are storable in the storage solutions of this invention for a period of at least about five days, more preferably at least about seven days.

Further provided is a method of storing a blood product comprising: adding to said blood product a solution comprising at least an amount effective to inhibit growth of a selected microorganism, of a compound selected from the group consisting of allicin, and microorganism-growth-inhibiting analogs and derivatives thereof; and storing said blood product.

A method of treating a patient in need of a blood product is also provided, comprising: providing a blood product; adding to said blood product a solution comprising at least an amount effective to inhibit growth of a selected microorganism, of a compound selected from the group consisting of allicin and microorganism-growth-inhibiting analogs and derivatives thereof; storing said blood product; and administering said blood product to a patient without removing said compound. Preferably the blood product is platelets which are stored for at least about five days, more preferably at least about seven days.

### BRIEF DESCRIPTION OF THE FIGURES

FIG. 1 is a graph showing the bacteriostatic effect of garlic extract on S. epidermidis in platelets

FIG. 2 is a graph showing the bacteriostatic effect of garlic extract on Y. entercolitica in trypticase soy broth (TSB).

FIG. 3A is a graph showing the bacteriostatic effect of garlic extract on 1-2 log E. coli in platelets. FIG. 3B is a graph showing the bacteriostatic effect of garlic extract on 2-3 logs E. coli in platelets.

FIG. 4A is a graph showing the bacteriostatic effect of garlic extract on 1.0×10⁴ Staph. aureus in plasma. FIG. 4B is a graph showing the bacteriostatic effect of garlic extract on 2.5×10² Staph. aureus in plasma.

FIG. 5 is a graph showing the bacteriostatic effect of garlic extract on Klebsiella pneumoniae in platelets spiked with 1 log/ml.

### DETAILED DESCRIPTION

Blood product storage solutions comprising garlic extract, allicin and analogs and derivatives are provided herein, as well as methods using such compositions to inhibit growth of microorganisms and thereby increase the safety and, in some cases, the storage life of blood products.

As defined herein, “pharmaceutical salts” are non-toxic salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

The compounds of this invention may contain an asymmetric carbon atom, and some of the compounds of this invention may contain one or more asymmetric centers, and may thus give rise to optical isomers and diastereomers. While shown without respect to stereochemistry in the above generic formulas, the present invention includes such optical isomers and diastereomers, as well as the racemic and resolved, enantiomerically pure R and S stereoisomers, as well as other mixtures of the R and S stereoisomers and pharmaceutically acceptable salts thereof.

The term “alkyl” takes its usual meaning in the art and is intended to include straight-chain, branched and cycloalkyl groups. The term includes, but is not limited to, methyl, ethyl, n-propyl, isopropyl, n-butyl, sec-butyl, isobutyl, tert-butyl, n-pentyl, neopentyl, 2-methylbutyl, 1-methylbutyl, 1-ethylpropyl, 1,1-dimethylpropyl, n-hexyl, 1-methylpentyl, 2-methylpentyl, 3-methylpentyl, 4-methylpentyl, 3,3-dimethylbutyl, 2,2-dimethylbutyl, 1,1-dimethylbutyl, 2-ethylbutyl, 1-ethylbutyl, 1,3-dimethylbutyl, n-heptyl, 5-methylhexyl, 4-methylhexyl, 3-methylhexyl, 2-methylhexyl, 1-methylhexyl, 3-ethylpentyl, 2-ethylpentyl, 1-ethylpentyl, 4,4-dimethylpentyl, 3,3-dimethylpentyl, 2,2-dimethylpentyl, 1,1-dimethylpentyl, n-octyl, 6-methylheptyl, 5-methylheptyl, 4-methylheptyl, 3-methylheptyl, 2-methylheptyl, 1-methylheptyl, 1-ethylhexyl, 1-propylpentyl, 3-ethylhexyl, 5,5-dimethylhexyl, 4,4-dimethylhexyl, 2,2-diethylbutyl, 3,3-diethylbutyl, and 1-methyl-1-propylbutyl. Alkyl groups are optionally substituted. Lower alkyl groups are C₁-C₆ alkyl and include among others methyl, ethyl, n-propyl, and isopropyl groups.

The term “cycloalkyl” refers to alkyl groups having a hydrocarbon ring, particularly to those having rings of 3 to 7 carbon atoms. Cycloalkyl groups include those with alkyl group substitution on the ring. Cycloalkyl groups can include straight-chain and branched-chain portions. Cycloalkyl groups include but are not limited to cyclopropyl, cyclobutyl, cyclopentyl, cyclohexyl, cycloheptyl, cyclooctyl, and cyclononyl. Cycloalkyl groups can optionally be substituted.

Aryl groups may be substituted with one, two or more simple substituents including, but not limited to, lower alkyl, e.g., methyl, ethyl, butyl; halo, e.g., chloro, bromo; nitro; sulfato; sulfonyloxy; carboxy; carbo-lower-alkoxy, e.g., carbomethoxy, carboethoxy; amino; mono- and di-lower-alkylamino, e.g., methylamino, ethylamino, dimethylamino, methylethylamino; amido; hydroxy; lower-alkoxy, e.g., methoxy, ethoxy; and lower-alkanoyloxy, e.g., acetoxy.

The term “unsaturated alkyl” group is used herein generally to include alkyl groups in which one or more carbon-carbon single bonds have been converted to carbon-carbon double or triple bonds. The term includes alkenyl and alkynyl groups in their most general sense. The term is intended to include groups having more than one double or triple bond, or combinations of double and triple bonds. Unsaturated alkyl groups include, without limitation, unsaturated straight-chain, branched or cycloalkyl groups. Unsaturated alkyl groups include without limitation: vinyl, allyl, propenyl, isopropenyl, butenyl, pentenyl, hexenyl, hexadienyl, heptenyl, cyclopropenyl, cyclobutenyl, cyclopentenyl, cyclopentadienyl, cyclohexenyl, cyclohexadienyl, 1-propenyl, 2-butenyl, 2-methyl-2-butenyl, ethynyl, propargyl, 3-methyl-1-pentynyl, and 2-heptynyl. Unsaturated alkyl groups can optionally be substituted. Double bonds may be cis or trans.

Substitution of alkyl, cycloalkyl and unsaturated alkyl groups includes substitution at one or more carbons in the group by moieties containing heteroatoms. Suitable substituents for these groups include but are not limited to OH, SH, NH₂, COH, CO₂H, OR_{c}, SR_{c}, P, PO, NR_{c}R_{d}, CONR_{c}R_{d}, and halogens, particularly chlorines and bromines where R_{c}, and R_{d}, independently, are alkyl, unsaturated alkyl or aryl groups. Preferred alkyl and unsaturated alkyl groups are the lower alkyl, alkenyl or alkynyl groups having from 1 to about 3 carbon atoms.

The term “aryl” is used herein generally to refer to aromatic groups which have at least one ring having a conjugated pi electron system and includes without limitation carbocyclic aryl, aralkyl, heterocyclic aryl, biaryl groups and heterocyclic biaryl, all of which can be optionally substituted. Preferred aryl groups have one or two aromatic rings.

“Aralkyl” refers to an alkyl group substituted with an aryl group. Suitable aralkyl groups include among others benzyl, phenethyl and picolyl, and may be optionally substituted. Aralkyl groups include those with heterocyclic and carbocyclic aromatic moieties.

The term “alkoxy group” takes its generally accepted meaning. Alkoxy groups include but are not limited to methoxy, ethoxy, n-propoxy, isopropoxy, n-butoxy, sec-butoxy, isobutoxy, tert-butoxy, n-pentyloxy, neopentyloxy, 2-methylbutoxy, 1-methylbutoxy, 1-ethyl propoxy, 1,1-dimethylpropoxy, n-hexyloxy, 1-methylpentyloxy, 2-methylpentyloxy, 3-methylpentyloxy, 4-methylpentyloxy, 3,3-dimethylbutoxy, 2,2-dimethoxybutoxy, 1-1-dimethylbutoxy, 2-ethylbutoxy, 1-ethylbutoxy, 1,3-dimethylbutoxy, n-pentyloxy, 5-methylhexyloxy, 4-methylhexyloxy, 3-methylhexyloxy, 2-methylhexyloxy, 1-methylhexyloxy, 3-ethylpentyloxy, 2-ethylpentyloxy, 1-ethylpentyloxy, 4,4-dimethylpentyloxy, 3,3-dimethylpentyloxy, 2,2-dimethylpentyloxy, 1,1-dimethylpentyloxy, n-octyloxy, 6-methylheptyloxy, 5-methylheptyloxy, 4-methylheptyloxy, 3-methylheptyloxy, 2-methylheptyloxy, 1-methylheptyloxy, 1-ethylhexyloxy, 1-propylpentyloxy, 3-ethylhexyloxy, 5,5-dimethylhexyloxy, 4,4-dimethylhexyloxy, 2,2-diethylbutoxy, 3,3-diethylbutoxy, 1-methyl-1-propylbutoxy, ethoxymethyl, n-propoxymethyl, isopropoxymethyl, sec-butoxymethyl, isobutoxymethyl, (1-ethyl propoxy)methyl, (2-ethylbutoxy)methyl, (1-ethylbutoxy)methyl, (2-ethylpentyloxy)methyl, (3-ethylpentyloxy)methyl, 2-methoxyethyl, 1-methoxyethyl, 2-ethoxyethyl, 3-methoxypropyl, 2-methoxypropyl, 1-methoxypropyl, 2-ethoxypropyl, 3-(n-propoxy)propyl, 4-methoxybutyl, 2-methoxybutyl, 4-ethoxybutyl, 2-ethoxybutyl, 5-ethoxypentyl, and 6-ethoxyhexyl.

A “dithiin” is a ring having two sulfur atoms in the ring, one double bond in the ring. appended to the ring.

“Optional” or “optionally” means that the subsequently described event or circumstance may or may not occur, and that the description includes instances where said event or circumstance occurs and instances in which it does not. For example, “optionally substituted phenyl” means that the phenyl radical may or may not be substituted and that the description includes both unsubstituted phenyl radicals and phenyl radicals wherein there is substitution.

### EXAMPLES

### Example 1. Preparation of Garlic Extract

Garlic extract was prepared by peeling cloves and storing them in a closed container. They were then mixed with 0.9% saline (25 g of cloves to 100 ml saline) in a blender until liquified, and centrifuged at 12,000 rpm. The supernatant was decanted and filtered through 0.2 μm filter (sterile barrier). The preparation was frozen and stored for use.

### Example 2. Preparation of Allicin

Allicin was prepared by the following process: One gram of fractionally distilled diallyl disulfide was dissolved in 5 ml cold (4° C.) glacial acetic acid. Then 1.5 ml cold 30% hydrogen peroxide was slowly added. After 30 minutes, the temperature was allowed to increase to 12-15° C. Stirring was continued for 4-6 hours until the diallyl disulfide content decreased by only 75-80% (avoiding oxidation to diallyl thiosulfonate). The reaction was stopped with 15 ml water and extracted with 30 ml dichloromethane. Acetic acid was removed by washing with 5% NaHCO₃ then water to pH 6-7. Solvent was evaporated and the material was redissolved in 500 ml water. Unreacted diallyl disulfide was removed by double extraction with 0.1 volume hexane.

### Example 3. Platelet Safety

Two ml of garlic extract (Example 1) were added to two-day old platelets to make the composition equivalent to about 1:100 dilution of the extract. Samples of the platelets were taken before the addition of the garlic extract, after, and the next day.

Swirl was maintained and at day 3 the platelets continued to metabolize O₂ (indicative by the PO₂ staying at about 80-95), and the pH was stable. Glucose was consumed and lactate produced at typical rates. Most importantly the cells remained viable and did not die. Garlic extract given to platelets at 1:100 extract:platelets had no measurable or visible effect on the platelets.

TABLE 2

| | Day 2 (pre-garlic) | Day 2 (post-garlic) | Day 3 |
|---|---|---|---|
| Swirl | 3 | 3 | 3 |
| Lactate | 6.06 | 5.95 | 7.86 |
| Glucose | 17.8 | 17.5 | 16.2 |
| pH | 7.13 | 7.23 | 7.16 |
| PO₂ | 86 | 91 | 84 |
| PCO₂ | 26 | 21 | 21 |
| Cell count | 1581 × 10³ | 1568 × 10³ | 1495 × 10³ |

### Example 4. Bacteriostatic Effects of Garlic Extraction Platelets on Staphylococcus epidermidis

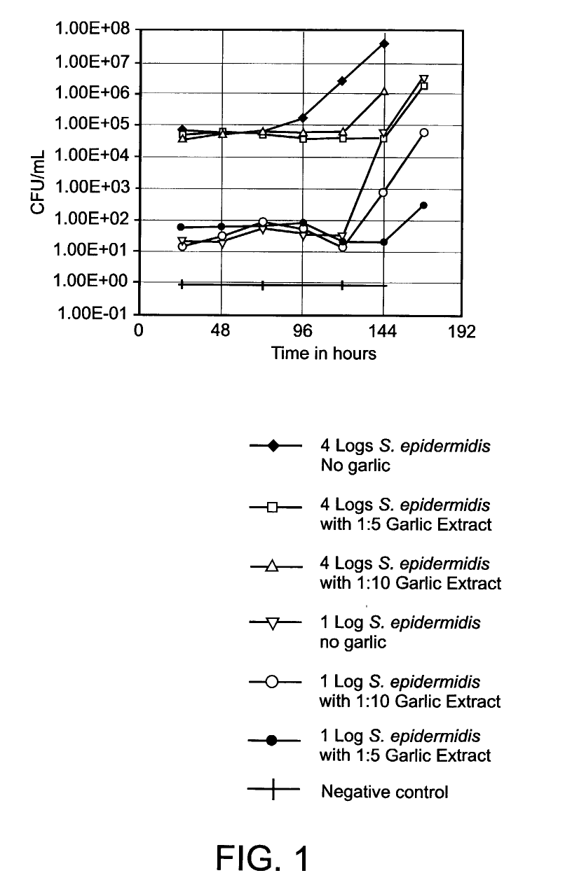

Garlic extract was prepared according to the procedure of Example 1. Staphylococcus epidermidis was inoculated into platelets at a 5.0×10⁴ and 5.0 10¹ titer. 25 ml samples were prepared having 1:5 garlic extract to platelets and 1:10 garlic extract to platelets. Samples were plated, grown and counted. Results are shown in FIG. 1.

### Example 5. Bacteriostatic Effects of Garlic Extract on S. epidermidis TSA

To test the ability of garlic extract to kill or halt the growth of S. epidermidis (gram positive bacteria), 1 ml. aliquots of garlic extract (Example 10 dilutions (from 1:1 to 1:1000) were mixed with 100 μl of S. epidermidis (1.19×10⁸) and plated on a trypticase soy agar (TSA) plate where they were allowed to grow for 24 hours at 37° C. A positive control without garlic extract was also plated. Visual inspection after 24 hours revealed that for undiluted garlic extract to 1:3 garlic extract:water dilutions, there was no bacterial growth at 24 hours. For dilutions of 1:4 to 1:10 there were fewer colonies and small colony size compared to control. Effects at dilutions of 1:100 to 1:1000 were negligible. In broth, garlic extract can prevent S. epidermidis from growing over a 96-hour period.

### Example 6. Bacteriostatic Effects of Garlic Extract on Yersinia entercolitica in Broth

Effect on Yersinia entercolitica growth in trypticase soy broth (TSB) was also tested. Three ml. aliquots of 5.0×10⁶ cfu/ml were mixed with garlic extract dilutions in cuvettes as follows:

Control—no garlic w/25 ml TSB

1:5 garlic—1.0 ml garlic w/4 ml TSB

1:10 garlic—0.5 ml garlic w/4.5 ml TSB

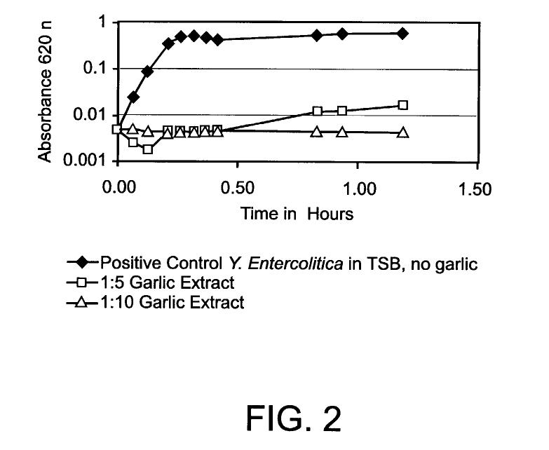

The cuvettes were vortexed, capped and sealed, and incubated in a 37° C. Rosi incubator at 120 rpm. Samples were spectrophotometrically observed over a 96-hour period. FIG. 2 shows results. Even at 1:10 dilution, Yersinia entercolitica growth is affected.

### Example 7. Bacteriostatic Effect of Garlic Extract on Escherichia coli in Platelets

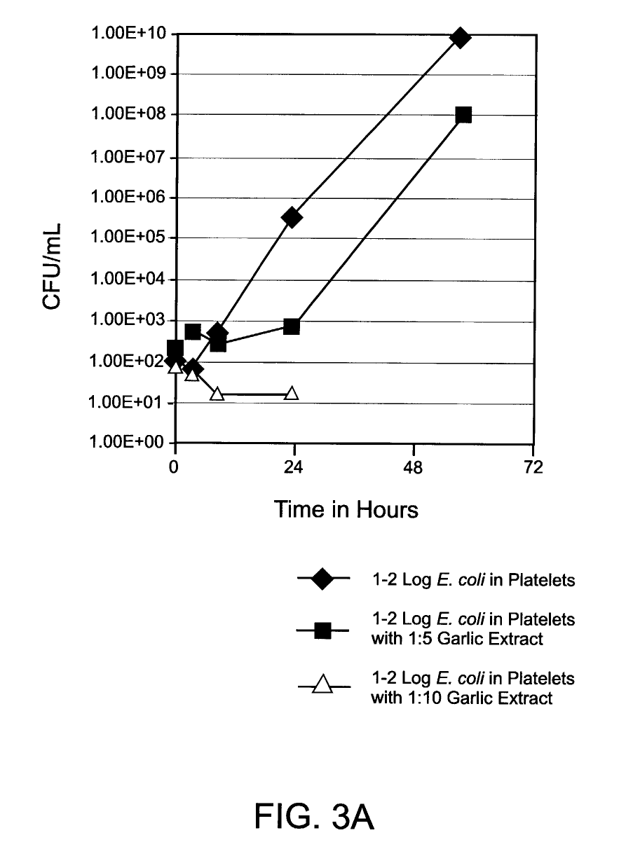

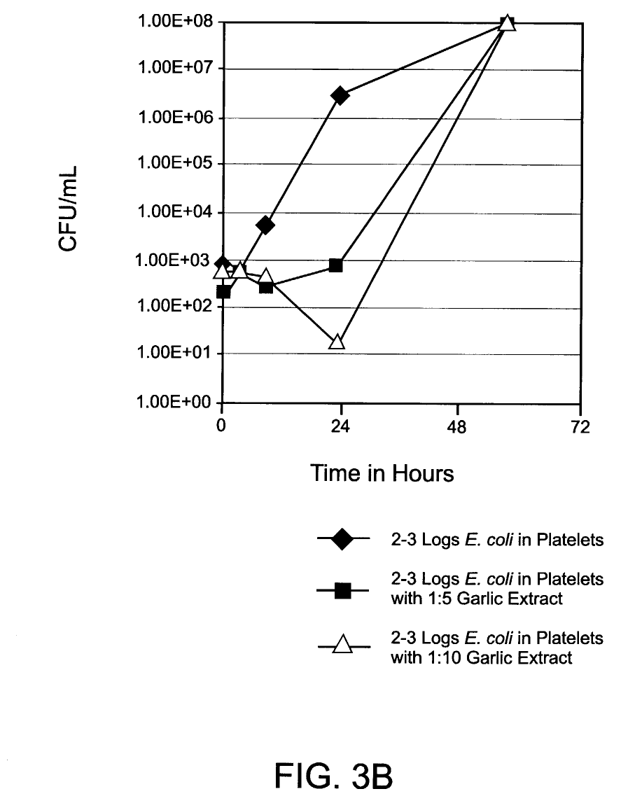

The procedure of Example 4 was followed to test the bacteriostatic effect of garlic extract on Escherichia coli with platelets, using approximate starting titers of 1 log and 2 logs of E. coli. Results are shown in FIGS. 3A and 3B.

### Example 8. Bateriostatic Effect of Garlic Extract on Staphylococcus aureus in Platelets

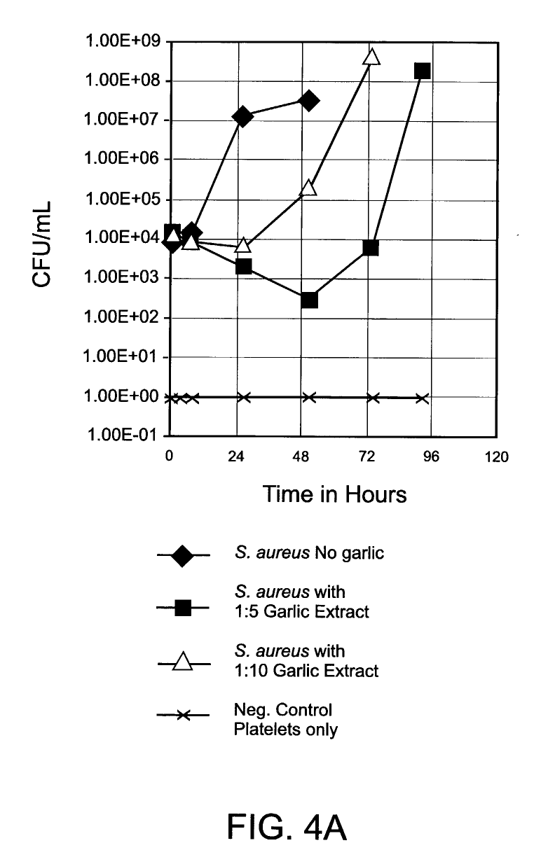

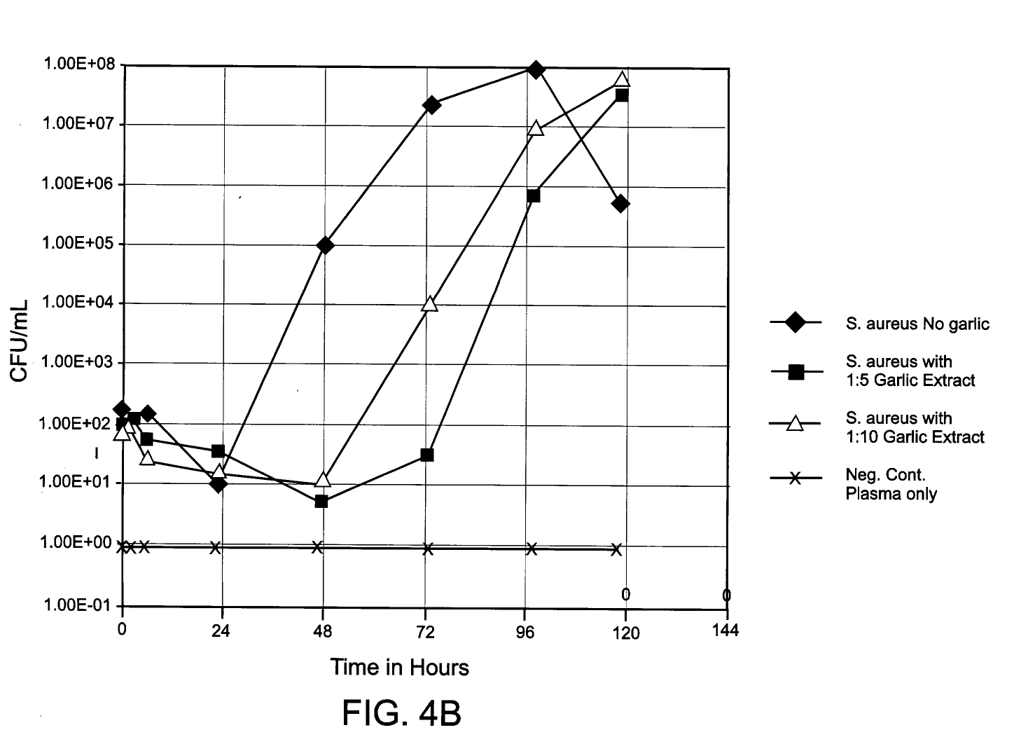

The procedure of Example 4 was followed to test the bacteriostatic effect of garlic extract on Staphylococcus aureus in platelets using a starting titer of ˜1.0×10⁴. Results are shown in FIG. 4A. The 1:10 preparation was bacteriostatic for 24 hours and the 1:5 preparation was bacteriostatic for 72 hours.

### Example 9. Bacteriostatic Effect of Garlic Extract on Staphylococcus aureus in Plasma

To test the bacteriostatic effect of garlic extract on Staphylococcus aureus in plasma, plasma was inoculated to a starting titer of ˜2.5×10². After incubation of sample containing no garlic extract, 1:5 garlic extract to plasma, and 1:10 garlic extract to plasma, results were assayed. Results are shown in FIG. 4B.

### Example 10. Bacteriostatic Effect of Garlic Extract on Klebsiella pneumoniae in Platelets

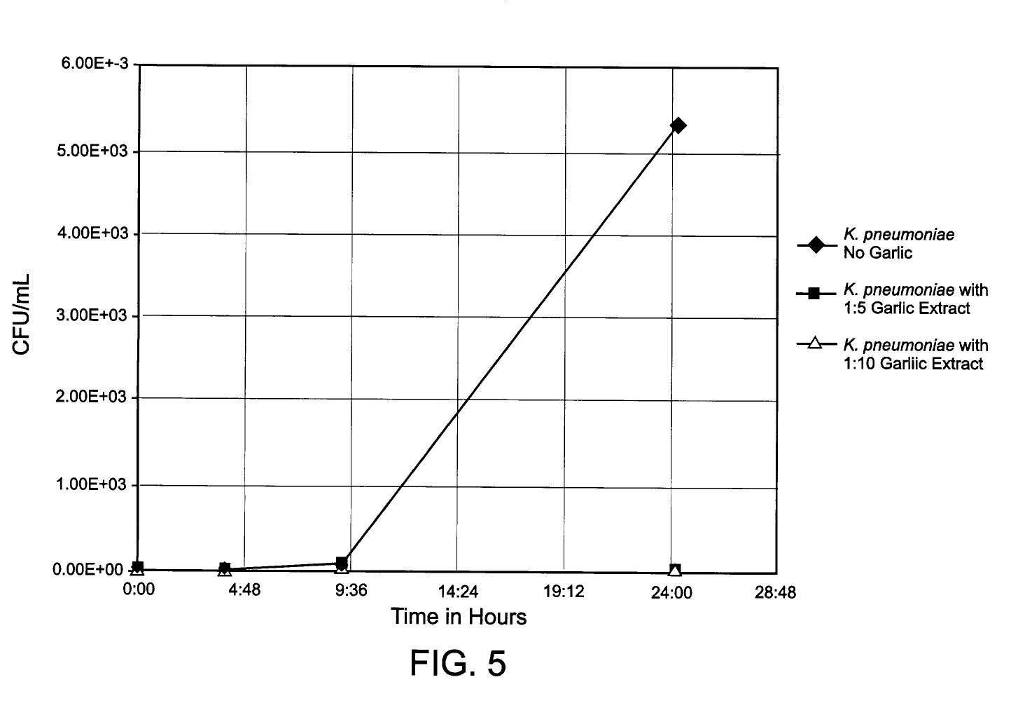

The procedure of Example 4 was followed to test the bacteriostatic effect of garlic extract on Klebsiella pneumoniae in platelets using a starting titer of 5.0×10². Results are shown in FIG. 5.

It will be readily understood by those skilled in the art that the foregoing description has been for purposes of illustration only and that a number of changes may be made without departing from the scope of the invention. For example, other compounds from allium species, and other allicin analogs and derivatives than those mentioned may be used, preferably those which are not toxic and do not have toxic breakdown products. The embodiments described herein are merely exemplary, and changes and modifications in the specifically-described embodiments can be carried out by one skilled in the art without departing from the scope of the invention. All such changes and modifications are intended to be included within the scope of the invention as defined in the appended claims.

1. An aqueous additive solution for blood products comprising a composition comprising a microorganism-growth-inhibiting compound derived from garlic or analog thereof, in an amount effective to inhibit growth of at least one selected microorganism.

2. The solution of claim 1 wherein said compound is present in an extract of garlic.

3. The blood product of claim 1 wherein said compound is allicin.

4. A blood product comprising the solution of claim 1 and whole blood.

5. The blood product of claim 4 comprising between about 65 volume percent and about 95 volume percent whole blood, and between about 5 volume percent and about 35 volume percent of said composition.

6. A blood product comprising the solution of claim 1 and red blood cells.

7. The blood product of claim 6 comprising between about 65 volume percent and about 95 volume percent red blood cells, and between about 5 volume percent and about 35 volume percent of said composition.

8. A blood product comprising the solution of claim 1 and white blood cells.

9. The blood product of claim 8 comprising between about 65 volume percent and about 95 volume percent white blood cells, and between about 5 volume percent and about 35 volume percent of said composition.

10. A blood product comprising the solution of claim 1 and platelets.

11. The blood product of claim 10 comprising between about 65 volume percent and about 95 volume percent white blood cells, and between about 5 volume percent and about 35 volume percent of said composition.

12. A blood product comprising the solution of claim 1 and serum.

13. The blood product of claim 12 comprising between about 65 volume percent and about 95 volume percent white blood cells, and between about 5 volume percent and about 35 volume percent of said composition.

14. The additive solution of claim 1 in which the blood product is platelets which have been stored for at least five days and remain viable.

15. The additive solution of claim 1 in which the blood product is platelets which have been stored for at least seven days.

16. The solution of claim 1 wherein said compound is selected from the group consisting of methyl propyl sulfide, allyl methyl sulfide, diallyl sulfide, methyl sulfide, diallyl disulfide, dimethyl disulfide, methyl propyl disulfide, dipropyl disulfide, allyl alcohol, allyl mercaptan, diallyl trisulfide, diallyl tetrasulfide, diallyl pentasulfide, diallyl hexasulfide, methy allyl disulfide, methyl allyl trisulfide, methyl allyl tetrasulfide, methyl allyl pentasulfide, methyl allyl hexasulfide, dimethyl disulfide, dimethyl trisulfide, dimethyl tetrasulfide, dimethyl pentasulfide, dimethyl hexasulfide, diethyl disulfide, propyl allyl disulfide, 2-vinyl-4H-1,3-dithiin, 3-vinyl-4H-1,2-dithiin, (E)-ajoene, (Z)-ajoene, allicin, allyl methyl thiosulfinate, (E)-2,3,7-trithiaocta-4-ene-7-oxide, (E)-4,5,9-trithiadeca-1,6-diene-9-oxide, (Z)-2,3,7-trithiaocta-4-ene-7-oxide, (Z)-4,5,9-trithiadeca-1,6-diene-9-oxide, (E)-4,5,9-trithiadeca-1,7-diene-9-oxide, (Z)-4,5,9-trithiadeca-1,7-diene-9-oxide, 2-propene-1-sulfinothioic acid S-(Z,E)-1-propenyl ester, 2-propenesulfinothioic acid S-methyl ester, and methanesulfinothioic acid S-(Z,E)-1-propenyl ester.

17. The solution of claim 1 wherein said compound is selected from the group consisting of compounds of the formula: 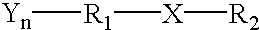
where:
R₁ and R₂ are, independently H, C₁-C₂₀ (saturated or unsaturated) alkyl, alkoxy, cycloalkyl or aralkyl;
X is S, SS, SSS, or SOS, and moieties in which one or more of the sulfur atoms are replaced with SO or OSO;
n is 0 or 1; and
Y is R₃ₘXR₄ₚ where
m and p are, independently, 0 or 1; and
R₃ and R₄ are H or C₁-C₆ (saturated or unsaturated) alkyl, alkoxy, cycloalkyl or aralkyl;
and
C₁-C₆ (saturated or unsaturated) dithiins;
and optical isomers and pharmaceutical salts thereof.

18. The solution of claim 17 wherein m and p are 1, R₃ is methyl, X is S═O and R₄ is

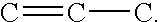

19. The solution of claim 1 wherein said compound is selected from the group consisting of compounds of the formula: 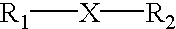
where
R₁ and R₂ are, independently H, or C₁-C₆ (saturated or unsaturated) alkyl;
and optical isomers and pharmaceutical salts thereof.

20. The solution of claim 19 wherein R₁ is allyl and R₂ is allyl ethyl, vinyl, or methyl.

21. The solution of claim 1 wherein isoalloxazine or an isoalloxazine analog is also added to said storage solution.

22. The solution of claim 1 where isoalloxazine or an isoalloxazine analog is added to said storage solution and said solution is exposed to photoradiation prior to addition to said storage solution of said garlic extract, allicin, other microorganism-growth-inhibiting compounds from garlic, and analogs and derivatives of allicin and said other compounds.

23. A method of reducing contamination of a blood product comprising:
adding to said blood product a solution comprising a composition comprising a microorganism-growth-inhibiting compound derived from garlic or analog thereof in an amount effective to inhibit growth of at least one selected microorganism.

24. A blood product having a prolonged storage life made by the method of claim 23.

25. The method of claim 23 wherein said solution also comprises isoalloxazine or an isoalloxazine analog.

26. A method of storing a blood product comprising:
a. adding to said blood product a solution comprising a composition comprising a microorganism-growth-inhibiting compound derived from garlic or analog thereof in an amount effective to inhibit growth of at least one selected microorganism; and
b. storing said blood product.

27. The method of claim 26 wherein said blood product is platelets and said method comprises storing said platelets for at least about five days wherein said stored platelets remain viable.

28. The method of claim 27 comprising storing said platelets for a period of at least about seven days wherein said stored platelets remain viable.

29. The method of claim 26 wherein said solution also comprises isoalloxazine or an isoalloxazine analog.

30. A method of treating a patient in need of a blood product comprising:
a. providing a blood product;
b. adding to said blood product a solution comprising a composition comprising a microorganism-growth-inhibiting compound derived from garlic or analog thereof in an amount effective to inhibit growth of at least one selected microorganism;
c. storing said blood product; and
d. administering said blood product to a patient without removing said compound.

31. The method of claim 30 wherein said blood product is platelets and is stored for a period of at least about five days and remains viable.

32. The method of claim 30 wherein said blood product is stored for a period of at least about seven days and cells therein remain viable.

33. The method of claim 30 wherein said solution also comprises isoalloxazine or an isoalloxazine analog.

34. A composition comprising an anticoagulant and a solution comprising a composition comprising a microorganism-growth-inhibiting compound derived from garlic or analog thereof in an amount effective to inhibit growth of at least one selected microorganism.

35. The composition of claim 34 also comprising a blood product.
